(19) United States
(12) Patent Application Publication
(10) Pub. No.: US 2008/0227853 A1
(43) Pub. Date: Sep. 18, 2008

(54) ANDROGENE MEMBRANE RECEPTOR AGONIST

(75) Inventor: Elias Castanas, Heraklion (GR)

Correspondence Address: SUGHRUE MION, PLLC, 2100 PENNSYLVANIA AVENUE, N.W., SUITE 800, WASHINGTON, DC, 20037, US

(73) Assignee: BIONATURE E. A. LIMITED, Egomi (CY)

(21) Appl. No.: 11/685,433

(22) Filed: Mar. 13, 2007

(51) Int. Cl. A61K 31/353; A61P 35/00; A61P 35/02

(52) U.S. Cl. 514/456

(57) ABSTRACT

Disclosed is a method of treating or preventing a disorder influenced by the action of a membrane androgen receptor, wherein a patient suffering or expected of suffering of such disorder is administered in pharmaceutically effective amounts an agonist of the membrane androgen receptor, said agonist being selected among the group consisting of natural, unnatural, oligorimerised or rearranged catechin, epicatechin, and derivatives thereof. The disease being treated or prevented may be cancer.

### INTRODUCTION

The present invention relates in general to the prevention and treatment of diseases, the etiology of said disease being caused or influenced by cells displaying an androgen membrane receptor on the membrane. Furthermore, the invention relates to a method comprising the step of administering an agonist selected among the group consisting of natural, unnatural, oligorimerised or rearranged catechin, epicatechin, and derivatives thereof to treat or prevent diseases influenced the action of an androgen membrane receptor.

### PRIOR ART

The membrane androgen receptor has been found to be preferentially expressed in a number of malignancies (eg. breast and prostate cancer, lung and pleural cancer, leukemias and lymphomas, ovarian and neural cancer). The activation of the receptor induces polymerization of the actin cytoskeleton and apoptosis.

Polyphenols belongs to a class of naturally occurring phenolic compounds found throughout the plant kingdom. Polyphenols are thus consumed daily in our vegetable diet in large amount.

In the literature, catechin and epicatechin have been found to be potent inhibitors of a number of cancer cell line proliferation (Damianaki et al., 2000; Kampa et al., 2000; Kampa et al., 2002a; Notas et al., 2005), interacting with different cellular mechanisms including cell growth arrest, cell cycle modulation and inhibition of Nitric Oxide Synthase (NOS) activity. Further, the focus of many other studies has been the effect of catechins on apoptosis, or programmed cell death. Much of the prior work in the art has attempted to determine what, if any, effect EGCg has on the growth inhibition and apoptosis induction of cancer cells. A differential growth inhibitory effect was reported in human colorectal cancer cells CaCo-2, breast cancer cells Hs578T, and their non-cancer cell counterparts (Ahmad and Mukhtar, 1999). EGCg has been implicated in the growth arrest and subsequent induction of apoptosis following cell growth inhibition has been shown in virally transformed fibroblast cells WI138, human epidermal carcinoma cells A431, lung cancer tumor cells H611, prostate cancer cell lines LNCaP, PC-3, and DU145, human carcinoma keratinocytes HaCaT, and mouse lymphoma cells LY—R (Ahmad et al., 1997; Chen et al., 1998; Yang et al., 1998; Ahmad and Mukhtar, 1999), trough a specific cancer cell action (Ahmad et al., 1997).

Catechins have also been shown to enhance the effect of cancer prevention drugs in vitro. For example, EGCg has been shown to enhance the apoptotic effect of sulindac and tamoxifen, presumably by EGCg enhancing the intracellular concentration of the cancer prevention drugs ((Suganuma et al., 1999), or inhibition of TNFa release (Piazza et al., 1995; Suganuma et al., 1996).

Proanthocyanadins are dietary sources of polyphenols. As described by Fine in a review article (Fine, 2000), they are naturally occurring plant metabolites widely available in fruits, vegetables, nuts, seeds, flowers, and bark and are primarily known for their antioxidant activity. Oligomeric proanthocyanidins are naturally occurring antioxidants widely available in fruits, vegetables, nuts, seeds, flowers and bark, which have been reported to possess a broad spectrum of biological, pharmacological and therapeutic activities against free radicals and oxidative stress. As described in Bagchi et al., a novel grape seed proanthocyanidin extract has been shown to provide excellent protection against oxidative stress and free radical-mediated tissue injury (Bagchi et al., 2000), while a polyphenolic fraction from grape seeds results in the inhibition of TPA-induced tumor promotion in CD-1 mouse epidermis (Bomser et al., 1999).

Membrane androgen receptors are a new class of membrane binding sites present preferentially on cancer cells. Indeed, our results show that these sites are found in prostate cell lines (Kampa et al., 2002b; Hatzoglou et al., 2004; Kampa et al., 2005), expressed preferentially on cancer rather than in non-neoplastic cells (Stathopoulos et al., 2003), in which they induce, after activation, actin cytoskeleton polymerization and redistribution (Kampa et al., 2002b; Papakonstanti et al., 2003), and apoptosis (Hatzoglou et al., 2004; Kampa et al., 2005). It is interesting to note that membrane androgen receptors may be different from intracellular androgen receptors, as they are not recognized by antibodies against the latter (Kampa et al., 2002b), and they are not inhibited by a series of commonly used antiandrogens in vitro (Papakonstanti et al., 2003) or in vivo (Hatzoglou et al., 2004).

In patent application WO2004006966 to Medexis S.A. we disclose the use of testosterone-protein conjugates, acting as agonists on membrane testosterone receptors, for the treatment of solid cancers and hematologic malignancies. Furthermore, we disclose the possibility of combining steroid-protein conjugates with other cytoskeleton acting agents in order to enhance their anti-cancer activity.

In patent application WO2004030440 it is disclosed that oligomeric epicatechin and catechin-derived procyanidins can be used as treatments for breast cancer growth through cell cycle arrest. In patent application WO03057201 it is claimed that epigallocatechin gallate, epicatechin gallate, epigallocatechin, epicatechin, and catechin, in association with ascorbic compounds, L-lysine and L-proline can be used for blocking melanoma, breast, colon, lung or brain cancer cell proliferation and metastasis, through interaction with the activity of matrix metaloproteases and plasmin. In patent applications US2004142048 and WO02067965 it is claimed that administering to a mammal of a composition comprising tea catechins adjunctively with a composition comprising proanthocyanadins, may treat rectal carcinoma, colon carcinoma, breast carcinoma, ovarian carcinoma, small cell lung carcinoma, colon carcinoma, chronic lymphocytic carcinoma, hairy cell leukemia, osophogeal carcinoma, prostate carcinoma, breast cancer, myeloma, and lymphoma, or their metastases, without or as an adjuvant therapy with other anticancer agents. The mode of action proposed is the interaction of catechins or procyanidins with a specific cancer-related isoform of the membrane NADH (hydroquinone) oxidase with protein disulfide-thiol isomerase activity (NOX).

In patent application WO03092613 it is suggested to use catechin multimers, and particularly substituted catechin multimers as carrier moieties for the delivery of nucleophilic and cationic bioactive therapeutic agents to target sites in vivo. In patent application CN1376464 is claimed that catechin plus curcumin may decrease cancer cell (LoVo) growth in vitro. In patent application WO9637201 is claimed that a preparation including saturated and unsaturated fatty acids, catechin gallates, their derivatives, and synthetic analogs, may act as an antiandrogen by inhibiting 5-alpha-reductase activity.

### SUMMARY OF THE INVENTION

The invention is based on the discovery that certain compounds inhibit the binding of an androgen on its membrane cognitive receptors. Thus, the present invention relates to a method of treating or preventing a disorder influenced by the action of a membrane androgen receptor, wherein a patient suffering or expected of suffering of such disorder is administered in pharmaceutically effective amounts an agonist of the membrane androgen receptor, said agonist being selected among the group consisting of natural, unnatural, oligorimerised or rearranged catechin, epicatechin, and derivatives thereof.

In the present document the term “catechin” or “epicatechin” comprises the natural products as well as their esterified derivatives (e.g. catechin or epicatechin gallate), as shown as in FIG. 1. They design also their C2-epimerized compounds (Ent-series), their rearranged compounds (i.e. protocatechuic acid) as well as their own ethers or esters derivatives. Any person trained in the art may extend this list to further substituted substances. They may be natural compounds, semi-synthetic or synthetic analogs.

The terms “condensed tannins” or “oligomerised catechines or epicatechines” refer to di-, tri-, tetra- and pentamers of natural as well as the unnatural (Ent-series) and rearranged catechin or epicatechin, in which the components are condensed through a 4-6, a 4-8 or any other interflavanolic linkages, known to those skilled in the art. The condensed tannins may be natural compounds, semi-synthetic or synthetic analogs.

The terms “membrane testosterone receptor” or “membrane androgen receptor” refer to a specific membrane entity (not yet characterized biochemically), which:

1. binds selectively and with high affinity to testosterone or another androgen,

2. is not recognized by commercially available anti-androgen receptor antibodies,

3. action is not inhibited by classical antiandrogens (ex. flutamide or cyproterone acetate).

### SHORT DESCRIPTION OF THE DRAWINGS

FIG. 1 discloses a non-exhaustive scheme of the catechin and epicatechin family.

FIG. 2 discloses the results obtained in the experiment reported in example 1.

FIG. 3 shows pictures of tissue treated with catechin and epicatechin as well as the dimers B2 and B5. The experiments resulting in the pictures are described in example 2.

### DETAILED DISCLOSURE OF THE INVENTION

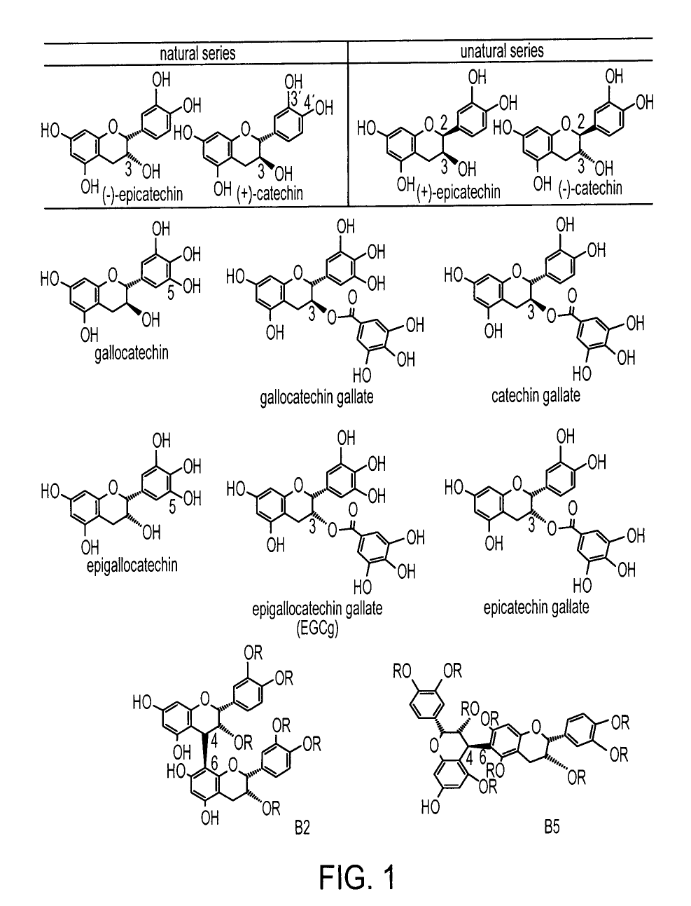

Natural, unnatural or rearranged catechin, epicatechin, the resulting oligomers (condensed tannins), and any of their derivatives are biogenetically issued from shikimate and polyketide pathways. Flavonoids form a peculiar family of polyphenols sharing the same C15-molecular framework and among those, is a special group, all featuring the 2-phenylbenzochromanol skeleton, known as flavanols (FIG. 1). In the series, the monomers are prone to form oligomers, also known as condensed tannins. The two main monomers, (+)-catechin and (−)-epicatechin may display modifications, for instance, to the hydroxylation or to the methoxylation patterns in the two 5 aromatic rings, and/or to the esterification or glycosylation of hydroxyle residues. These variations increase the number of possible derivatives largely. Examples of derivatives include: gallocatechin, gallocatechin gallate, epigallocatechin, epigallocatechin galtate (EGCg) and epicatechin gallate.

In an aspect of the invention the oligomer is a dimer having the general formalae:

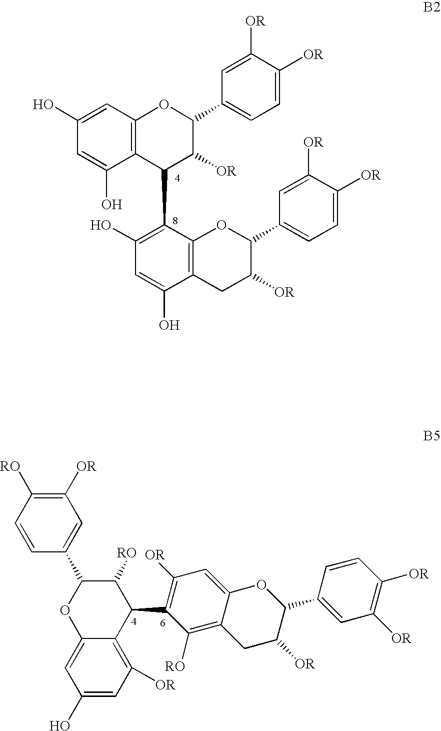

in which R independently is selected among the group consisting of H, C1-C12-alkyl, C2-C12-alken, C2-C12-alkyn, or gallic acid.

In an aspect of the invention the agonist is a specific displacer of testosterone from its membrane receptor sites. Thus, the affinity of these agonists used in the method of the invention is comprised in the nanomolar range. In another aspect, the invention claims that activation of testosterone membrane receptors by any of the natural, unnatural or rearranged catechin, epicatechin and their condensed tannins, induces a cytoskeletal alteration similar to that induced by testosterone-protein conjugates, in living cancer cells. The testosterone-protein conjugates may be prepared as disclosed in WO2004006966.

In a further aspect, it is found that all intermediate steps of signaling between the initial binding to membrane androgen receptors of natural, unnatural or rearranged polyphenols and their condensed tannins, and the final effect, i.e. actin polymerization are similar to those found after testosterone-BSA binding to membrane androgen receptors.

In a further aspect, it is claimed that administration of natural, unnatural or rearranged polyphenols or their condensed tannins in vivo might be beneficial for the management of malignancies in which the expression of testosterone membrane receptors can be detected. Such malignancies include breast, ovarian, prostate, lung, neural-crest derived cancers (the list is not exhaustive, and any person skilled in the art can expand it to any other cancer in which testosterone membrane receptors can be identified. In addition other malignancies such as leukemias and lymphomas can be also be treated.

In a final aspect, we claim that the agonist used in the method of the invention may be co-administered with other established cancer chemotherapeutic agents or their mixtures, in order to enhance their activity, to limit their adverse reactions or to restrain the administrated concentrations. Preferential chemotherapeutic agents include, but are not limited to adriamycin and adriamycin conjugates, mechlorethamine, cyclophosphamide, ifosfamide, melphalan, chlorambucil, hexamethylmelamine, thiotepa, busulfan, carmustine, lomustine, semustine, streptozocin, dacarbazine, methotrexate, fluorouacil, floxuridie, cytarabine, mercaptopurine, thioguanine, pentostatin, vinblastine, vincristine, velbanamine, vinflunine, etoposide, teniposide, actinomycin D, daunorubicin, doxorubicin, bleo-mycin, plicamycin, mitomycin, L-asparaginase, interferon-alpha, cisplatin, carboplatin, mitoxantrone, hydroxyurea, procarbazine, mitotane, aminoglutethimide, prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, acetogenins, e.g., bullatacin, and quassanoids, e.g. simalikalactone D and glaucarubolone, and pharmaceutically acceptable derivatives thereof.

The agonist used in the method of the invention may also be co-administered with cytoskeleton acting agents. The term “cytoskeleton acting agents” is used in the present document in their usual meaning. As examples of a cytoskeleton-acting drug can be mentioned Taxol® or Taxotere®. The combination of the agonist with cancer chemotherapeutic agents or their mixtures may be performed by the administration of one pharmaceutical composition comprising both type of agents, or may be performed by administration of separate pharmaceutical entities, one comprising the agonist and the other comprising the chemotherapeutic agent or any mixtures of.

Any mode of administration of the agonist and the optional chemotherapeutic agent be used, comprising per os, either as a native agent or after microencapsulation of the substance or by incorporating them in foods, or parenterally (hypodermal, intramuscular, intravenous). Any person skilled in the art may extend the mode of administration.

The dosages and regimens are generally to be determined in accordance with the discretion of the attending physician, taking due considerations to the patient's age, weight, condition etc.

Generally the daily dosages may be in the range of 1 mg/kg body weight to 100 mg/kg body weight, preferably in the range of 5 mg/kg body weight to 100 mg/kg body weight, more preferred in the range of 5 mg/kg body weight to 50 mg/kg body weight and most preferred in the range of 5 mg/kg body weight to 20 mg/kg body weight, and in a particular preferred embodiment the daily dosage is around 7 to 10 mg/kg body weight. The treatment is generally continued for up to 6 months, preferably in the range of 2 weeks to 6 months, more preferred in the range of 2 weeks to 3 months.

The pharmaceutical composition comprising the agonist and optionally chemotherapeutic agent may be administered at regular intervals during the period of treatment in order to maintain a satisfactory concentration of the active compound in the circulation, as the skilled person will appreciate. Thus, the pharmaceutical composition according to the invention may be administered once or more times daily or even with regular intervals of one or more days e. g. every second day, according to the discretion of the attending physician taking due consideration to the treatment efficiency and the acceptance of the patient being treated.

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories, or capsules. Further dosage forms are, for example, ointments, creams, pastes, foams, tinctures, lip-sticks, drops, sprays, dispersions, etc. Examples are capsules containing from about 0.05 g to about 1.0 g active ingredient.

The pharmaceutical compositions comprising the agonist for use in accordance with the present invention are prepared in a manner known per se, for example by means of conventional mixing, granulating, coating, dissolving or lyophilizing processes.

In an aspect of the invention, preference is given to the use of solutions of the active ingredient(s), and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80(R) (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinyl-pyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropyl-methylcellulose phthalate. Dyes or pigments may be added to the tablets or tablet coatings, for example for identification purposes or to indicate different doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

Pharmaceutical compositions suitable for rectal administration are, for example, suppositories that consist of a combination of the active ingredient and a suppository base. Suitable suppository bases are, for example, natural or synthetic triglycerides, paraffin hydrocarbons, polyethylene glycols or higher alkanols.

For parenteral administration, aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Solutions such as are used, for example, for parenteral administration can also be employed as infusion solutions. Preferred preservatives are, for example, antioxidants, such as ascorbic acid, or microbicides, such as sorbic acid or benzoic acid.

In an aspect of the invention, the agonist is microencapsulated with one or more compounds selected from cellulose hydroxypropyl ethers; low-substituted hydroxypropyl ethers; cellulose hydroxypropyl methyl ethers; methylcellulose polymers; ethylcelluloses and mixtures thereof; polyvinyl alcohol; hydroxyethylcelluloses; carboxymethylcelluloses and salts of carboxymethylcelluloses; polyvinyl alcohol and polyethylene glycol co-polymers; monoglycerides; triglycerides; polyethylene glycols, modified food starch, acrylic polymers; mixtures of acrylic polymers with cellulose ethers; cellulose acetate phthalate; sepifilms, cyclodextrins; and mixtures thereof.

### EXAMPLES

### Example 1

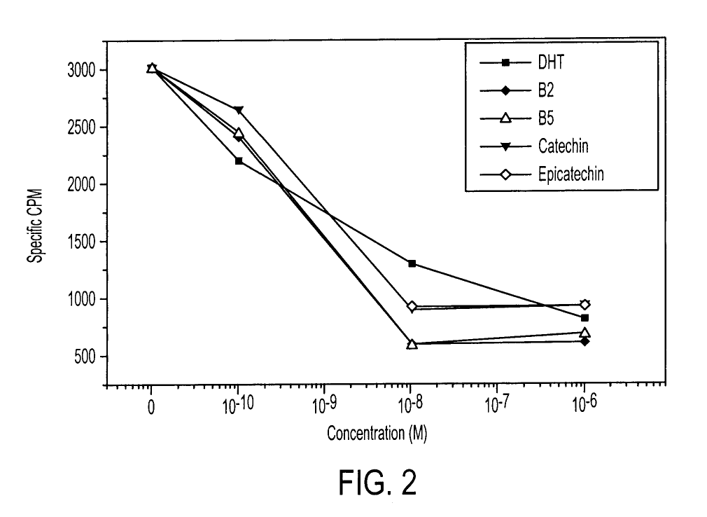

Membranes of T47D human breast cancer cells were treated with acid in order to dissociate any endogenous bound substance. Then the membranes were allowed to react with the radiolabeled testosterone. FIG. 2 presents the displacement of radiolabeled testosterone from the acid treated membranes by catechin, epicatechin, B2 and B5 dimers. The results are expressed as a percentage of specific binding.

### Example 2

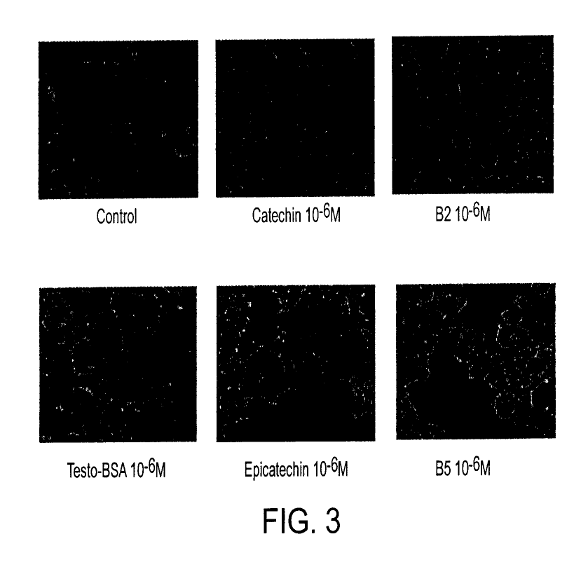

T47D human breast cancer cells were incubated with catechin, epicatechin B2 and B5 dimers for 24 hours and the actin in the cells were subsequently stained by rhodamine-phalloidin. FIG. 3 shows the modifications of the actin cytoskeleton (polymerization and sub-membrane redistribution).

### REFERENCES

Ahmad N, Feyes D K, Nieminen A L, Agarwal R and Mukhtar H (1997) Green tea constituent epigallocatechin-3-gallate and induction of apoptosis and cell cycle arrest in human carcinoma cells. J Natl Cancer Inst 89:1881-1886.

Ahmad N and Mukhtar H (1999) Green tea polyphenols and cancer: biologic mechanisms and practical implications. Nutr Rev 57:78-83.

Bagchi D, Bagchi M, Stohs S J, Das D K, Ray S D, Kuszynski C A, Joshi S S and Pruess H G (2000) Free radicals and grape seed proanthocyanidin extract: importance in human health and disease prevention. Toxicology 148:187-197.

Bomser J A, Singletary K W, Wallig M A and Smith M A (1999) Inhibition of TPA-induced tumor promotion in CD-1 mouse epidermis by a polyphenolic fraction from grape seeds. Cancer Lett 135:151-157.

Chen Z P, Schell J B, Ho C T and Chen K Y (1998) Green tea epigallocatechin gallate shows a pronounced growth inhibitory effect on cancerous cells but not on their normal counterparts. Cancer Lett 129:173-179.

Damianaki A, Bakogeorgou E, Kampa M, Notas G, Hatzoglou A, Panagiotou S, Gemetzi C, Kouroumalis E, Martin P M and Castanas E (2000) Potent inhibitory action of red wine polyphenols on human breast cancer cells. J. Cell Biochem. 78:429-441.

Fine A M (2000) Oligomeric proanthocyanidin complexes: history, structure, and phytopharmaceutical applications. Altern Med Rev 5:144-151.

Hatzoglou A, Kampa M, Kogia C, Charalampopoulos I, Theodoropoulos P, Anezinis P, Dambaki C, Papakonstanti E, Stathopoulos E, Stournaras C, Gravanis A and Castanas E (2004) Membrane androgen receptor activation induces apoptotic regression of human prostate cancer cells in vitro and in vivo. J Clin Endocrinol Metab [e-pub ahead of print]:PMID: 15585562.

Kampa M, Hatzoglou A, Notas G, Damianaki A, Bakogeorgou E, Gemetzi C, Kouroumalis E, Martin PM and Castanas E (2000) Wine antioxidant polyphenols inhibit the proliferation of human prostate cancer cell lines. Nutr. Cancer 37:223-233.

Kampa M, Nifli A, Charalampopoulos I, Alexaki V, Theodoropoulos P, Stathopoulos E, Gravanis A and Castanas E (2005) Opposing effects of estradiol- and testosterone-membrane binding sites on T47D breast cancer cell apoptosis. Exp Cell Res: (accepted).

Kampa M, Nistikaki A, Hatzoglou A, Kouimtzoglou H, Boskou D, Vercauteren J, Gravanis A and Castanas E (2002a) Phenolic acids and polyphenols are potent inhibitors of cancer cell proliferation: Assessment and mechanisms of action, in Polyphenols 2002: Recent advances in polyphenols research. (El-Hadrami I and Daayf F eds) pp 62-93, Groupe Polyphenols.

Kampa M, Papakonstanti E A, Hatzoglou A, Stathopoulos E N, Stournaras C and Castanas E (2002b) The human prostate cancer cell line LNCaP bears functional membrane testosterone receptors that increase PSA secretion and modify actin cytoskeleton. Faseb J 16:1429-1431.

Notas G, Kampa M, Nifli A, Vercauteren J, Kouroumalis E and Castanas E (2005) The system NO/NOS mediates the antiproliferative effects of wine polyphenols in HepG2 human hepatocellular carcinoma cell line. BMC Cell Biology: (accepted).

Papakonstanti E A, Kampa M, Castanas E and Stournaras C (2003) A rapid, nongenomic, signaling pathway regulates the actin reorganization induced by activation of membrane testosterone receptors. Mol Endocrinol 17:870-881.

Piazza G A, Rahm A L, Krutzsch M, Sperl G, Paranka N S, Gross P H, Brendel K, Burt R W, Alberts D S, Pamukcu R and et al. (1995) Antineoplastic drugs sulindac sulfide and sulfone inhibit cell growth by inducing apoptosis. Cancer Res 55:3110-3116.

Stathopoulos E N, Dambaki C, Kampa M, Theodoropoulos P A, Anezinis P, Delakas D, Delides G S and Castanas E (2003) Membrane androgen binding sites are preferentially expressed in human prostate carcinoma cells. BMC Clin Pathol 3:1.

Suganuma M, Okabe S, Kai Y, Sueoka N, Sueoka E and Fujiki H (1999) Synergistic effects of (−−)-epigallocatechin gallate with (−−)-epicatechin, sulindac, or tamoxifen on cancer-preventive activity in the human lung cancer cell line PC-9. Cancer Res 59:44-47.

Suganuma M, Okabe S, Sueoka E, Iida N, Komori A, Kim S J and Fujiki H (1996) A new process of cancer prevention mediated through inhibition of tumor necrosis factor alpha expression. Cancer Res 56:3711-3715.

Yang G Y, Liao J, Kim K, Yurkow E J and Yang C S (1998) Inhibition of growth and induction of apoptosis in human cancer cell lines by tea polyphenols. Carcinogenesis 19:611-616.

1. A method of treating or preventing a disorder influenced by the action of a membrane androgen receptor, wherein a patient suffering or expected of suffering of such disorder is administered in pharmaceutically effective amounts an agonist of the membrane androgen receptor, said agonist being selected among the group consisting of natural, unnatural, oligorimerised or rearranged catechin, epicatechin, and derivatives thereof.

2. A method according to claim 1, wherein the agonist is selected among the group comprising (−)-epicatechin, (+)-catechin, (+)-epicatechin, (−)-catechin, gallocatechin, gallocatechin gallate, catechin gallate, epigallocatechin, epigallocatechin gallate (EGCg), epicatechin gallate, and oligomers thereof.

3. The method according to claim 2, wherein the oligomer is a dimer having the general formalae: 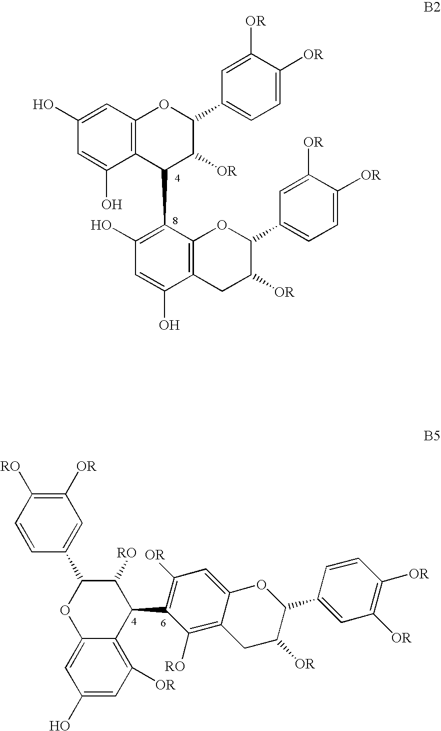
in which R independently is selected among the group consisting of H, C1-C12-alkyl, C2-C12-alken, C2-C12-alkyn, or gallic acid.

4. A method according to claim 1, wherein the androgen membrane receptor is membrane testosterone receptor.

5. A method according to claim 1, wherein the disorder being treated or prevented is cancer.

6. A method according to claim 5, wherein the cancer involves a solid tumor.

7. A method according to claim 5, wherein the cancer is a breast, prostate, lung, pleural, ovarian, or neural cancer, or a hematologic malignancy.

8. A method according to claim 7, wherein the hematologic malignancy is leukemia or lymphoma.

9. A method according to claim 1, wherein activation of the membrane androgen receptor induces apoptosis.

10. A method according to claim 1, wherein the treatment or prevention of the disorder influenced by the action of a membrane androgen receptor, comprises the additional administering of a chemotherapeutic agent.

11. A method according to claim 10, wherein the chemotherapeutic agent is a cytoskeleton modifying reagent.

12. A method according to claim 1, wherein the administered agonist of the androgen membrane receptor is microencapsulated.

13. A method according to claim 1, wherein the agonist and the optional chemotherapeutical agent is administered per orally, intravenously, subcutaneously, or intramuscularly.
